(19) United States
(12) Patent Application Publication
(10) Pub. No.: US 2008/0038406 A1
(43) Pub. Date: Feb. 14, 2008

(54) Method for Making a Liquid Concentrate of Food-Grade Acclimated and Viable Bacteria

(75) Inventors: Luc Terragno, Paris (FR); Guillaume Catonnet, Massy (FR); Pascal Regulier, Guyancourt (FR); Christophe Daval, Choisy Le Roi (FR); Philippe Teissier, Massy (FR); Jean-Yves Barbeau, Igny (FR)

Correspondence Address: FOLEY AND LARDNER LLP;SUITE 500, 3000 K STREET NW, WASHINGTON, DC, 20007, US

(73) Assignee: Compagnie Gervais Danone

(21) Appl. No.: 10/590,658

(22) PCT Filed: Feb. 28, 2005
(86) PCT No.: PCT/FR05/00479

§ 371 Date: Nov. 29, 2006

(30) Foreign Application Priority Data
Feb. 27, 2004 (FR) 0401999

(51) Int. Cl. A23L 1/48; C12M 1/12; C12N 1/20; C12Q 1/02

(52) U.S. Cl. 426/61; 435/252.1; 435/29; 435/297.1

(57) ABSTRACT

A method for making a liquid concentrate of food-grade acclimated and viable bacteria, preferably but not exclusively lactic acid bacteria.

The present invention relates to a production process of a liquid concentrate of adapted and viable bacteria for use in foodstuffs. Preferably but not limiting, the bacteria produced are lactic bacteria.

The ingestion of certain strains of bacteria, in particular those belonging to Lactobacillus and Bifidobacterium genera are particularly beneficial to health, especially by promoting proper functioning of the intestinal flora. In fact, these bacteria produce bacteriocines and of the lactic acid which boost the digestibility of foodstuffs, promote intestinal peristalsis, and accelerate the evacuation of plates. In addition, these bacteria produce certain B complex vitamins, and in general promote the absorption of vitamins and minerals, reduce blood cholesterol, reinforce the immune system and cover intestinal mucous to protect against invasion and activities of harmful microorganisms.

Because of this, for several years now, the agro-food industries have been attempting to incorporate such bacteria into their finished products, most generally yoghurts.

Currently, these bacteria are produced commercially, in a frozen or lyophilised form. However, these production processes are traumatising for the bacteria which lose part of their activity and at times their viability. This is prejudicial for industrial producers and for the consumers of these products since the bacteria must satisfy quality and technological performance requirements, if possible over several months. It would therefore be preferable to produce the bacteria by a process ensuring their viability and maximum activity. To this end, one method consists of producing the bacteria in a liquid form. However it has been revealed that this method also generates significant mortality among the bacteria, after the introduction of bacteria to the finished product.

In addition, to reduce storage costs of bacteria and ease addition of bacteria to the finished product, it should be desirable to concentrate the bacteria in liquid form. For this, the specialist normally utilises a centrifuging or filtration step. However, centrifuging is a traumatising process for the bacteria, and can cause considerable cellular mortality especially due to strong chiselling and also this process is not well adapted for centrifuging low volumes such as those required in the production of bacteria to be added as probiotics to food products. With respect to a classic filtration step, this also poses problems of mortality of bacteria and clogging des filters by the bacteria.

It would thus be desirable to produce a wanted volume of liquid concentrate of bacteria having maximum activity and viability after the concentration step and after introduction to the finished product.

Surprisingly and unexpectedly, the inventors have shown that an adaptation step of the bacteria helped significantly increase the activity and viability of the bacteria after introduction to the finished product.

In addition, the inventors have shown that a tangential filtration step, under certain conditions (pressure, concentration, membrane porosity, etc), helped concentrate the desired volumes of bacteria culture, while retaining their viability and without clogging of the filters.

Tangential filtration produces two currents as a function of the nature and structure of the membrane: the permeate (the culture medium substantially exempt from bacteria) and the residue (containing the bacteria, also called concentrate). In tangential filtration, the fluid circulates not perpendicularly but parallel to the surface of the membrane and its flow speed thus ensures autocleaning, preventing the accumulation of deposits blocking the filtration surface (commonly known as clogging the filters).

An object of the present invention is thus a production process of a liquid concentrate of adapted and viable bacteria, for use in foodstuffs comprising the following successive steps:
a) the bacteria are propagated in a fermenter in an appropriate culture medium;
b) the bacteria obtained are adapted to step a);
c) the culture medium containing the bacteria adapted by tangential microfiltration is washed using a washing solution;
d) the washed medium containing the bacteria adapted by tangential microfiltration to a bacterial concentration greater than 5.10¹⁰ ufc/ml advantageously greater than 1.10¹¹ ufc/ml are concentrated in bacteria;
e) a liquid concentrate of adapted and viable bacteria for use in foodstuffs is recovered.

According to the present invention the term bacteria is understood to preferably designate lactic bacteria, of Lactobacillus spp., Bifidobacterium spp., Streptococcus spp, Lactococcus spp. and in particular Lactobacillus casei, Lactobcacillus plantarum, Lactobacillus bulgaricus, Lactobacillus helveticus, Lactobacillus acidophilus, Bifidobaeterium animalis, Bifidobacterium breve, Streptococcus thermophilus, Lactococcus lactis genera.

Adapted bacteria is understood to designate, according to the present invention, bacteria more resistant to different stresses, in particular those associated with different physicochemical stresses.

According to the present invention the culture medium of step a) is a synthetic medium.

Synthetic medium is understood to designate according to the present invention a medium to which are introduced compounds subjected to rigorous quantitative and qualitative control.

According to the present invention, the washing solution is adapted to foodstuff utilisation of the bacteria concentrate, and presents an osmotic pressure compatible with the viability of the bacteria.

According to the present invention the culture medium, containing the bacteria in the fermenter at the end of step a), has a pH between 3 and 6.

According to the present invention, the bacteria concentration, at the end of propagation step a), is greater than 2.10¹⁰ ufc/ml.

In addition, the inventors have shown that adaptation of the bacteria conducted at step b) helps reduce the mortality of the bacteria caused by the change in medium of the bacteria, between their culture medium and the finished food product to be added.

According to the present invention adaptation of the bacteria is demonstrated by measuring parameters of the culture medium. According to the present invention, the parameters of the culture medium are preferably the pH, osmotic pressure and/or temperature.

Other parameters for revealing the adaptation of the bacteria are possible, such as for example the sugar concentration of the bacterian medium.

In the event where the parameter of the culture medium is the pH, step b) is preferably carried out by decreases in the pH by natural acidification.

In order to conduct the adaptation step of the bacteria at pH by natural acidification the sugar concentration of the fermentation medium can for example be measured, and beyond a threshold concentration for each species of bacteria, it is known that the pH is no longer regulated and adaptation to the medium becomes very easy.

So for example, if the sugar concentration of the fermentation medium of Lactobacillus casei is 9 g/l, the pH is no longer regulated and is approximately equal to 5. It then becomes easier for the adapted strain to be added to a new medium and this allows greater viability of the bacteria in the final medium.

According to the present invention, the parameter of the bacteria is the size of the bacteria.

In the event where the adaptation is disclosed by the size of the bacteria, the distribution of the lengths of each bacterium of said concentrate is preferably and predominantly between 0.1 and 10 micrometres, advantageously between 0.5 and 5 micrometres.

The size of the bacteria is measured by adapted means.

Adapted means can be for example regular sampling of bacteria followed by measuring the size of the bacteria by flux cytometry.

In addition, according to the present invention, tangential filtration can be utilised for step b) for adaptation of the bacteria.

According to the present invention the tangential filtration membranes have a porosity between 0.01 et 0.5 μm and preferably, between 0.1 and 0.3 μm.

These membranes are utilised for steps c) and d) of the process and optionally step b).

The filtration membranes are characterised by:
porosity and the thickness of the filtering layer on which the permeate rate depends.
the diameter of the pores and their distribution on which the efficacy of separation depends.
the material employed on which the mechanical, chemical and thermal resistance and the ease of cleaning depend.

Filtration membrane is understood to designate organic or mineral membranes.

Organic membranes can be composed inter alia of cellulose acetate, aromatic polyamides, polysulphone, cellulose esters, cellulose, cellulose nitrate, PVC, or polypropylene.

Mineral membranes can be composed inter alia of sintered ceramic, sintered metal, carbon, or glass.

According to the present invention the culture medium containing the bacteria is maintained at a temperature between 25 and 45° C., and preferably between 35 and 39° C.

According to the present invention the temperature is decreased by 1 to 44° C. at step b) so as to adapt the strain to the temperature of the finished product where they are to be added.

According to the present invention, at step c) the entry pressure of the culture medium in the filtration module is between 0 and 3.10⁵ Pa.

According to the present invention, in steps c) and d) the permeate rate is between 0.001 and 0.1 m³/h/m² of exchange surface.

According to the present invention in step d), the transmembrane pressure is between 0.1.10⁵ and 2.10⁵ Pa, preferably between 0.1.10⁵ and 0.5.10⁵ Pa and advantageously between 0.1.10⁵ and 0.5.10⁵ Pa.

The membrane is presented as a pure mechanical barrier allowing the components of a size less than the diameter of the pores to pass through. The separation between the two liquid phases is gained by applying a difference in pressure between the side where the culture medium containing the bacteria circulates and that where the permeate circulates (the culture medium substantially exempt of bacteria). This difference in pressure is commonly called transmembrane pressure.

Recirculation of the culture medium comprising the bacteria, in closed loop, in the tangential filtration module allows concentration of the bacteria and filtration of the culture medium through the membrane, limiting clogging.

According to the present invention in step d), the recirculation rate of the washed medium is between 0.5 and 3 m³/h/m² of exchange surface and advantageously between 0.8 and 1.25m³/h/m² of exchange surface.

According to the present invention the production process of a liquid concentrate of adapted and viable bacteria comprises prior to step a) the successive steps of revival and preculture of the bacteria.

To reduce to a maximum the latency phase in the fermenter, the microorganism is utilised in full exponential growth phase. To do this, the inventors proceed in two steps:
Execution of revival in a tube of bacteria previously frozen at −80° C.
Erlenmeyer preculture serving to multiply the number of microorganisms. Their growth should be stopped in the maximum exponential growth phase.

According to the present invention, the production process of a liquid concentrate of adapted and viable bacteria comprises an additional step f), after step e), of packaging into flexible, hermetic and sterile bags of the liquid concentrate of adapted and viable bacteria.

Flexible hermetic bags are understood to designate according to the present invention bags preferably made of foodstuff plastic.

According to the present invention, the process can comprise an additional step g) after the optional step f) of keeping the liquid concentrate of adapted and viable bacteria packaged in flexible and hermetic bags at low temperatures of between −50° C. to +4° C.

By way of option, it is possible to add to the liquid concentrate of adapted and viable bacteria packaged in flexible bags, and kept at low temperatures, cryoprotective molecules such as saccharose, for example.

According to the present invention, the process can comprise an additional step h), after step g), of reheating by adapted means of said liquid concentrate of adapted and viable bacteria packaged in flexible and hermetic bags.

Adapted means is understood to designate for example according to the present invention the utilisation of a bain marie at a temperature not lethal for the bacteria, for example 37° C.

An object of the present invention is also a device for carrying into effect the production process of a liquid concentrate of adapted and viable bacteria for use in foodstuffs according to the present invention, characterised in that it comprises a vat (1) containing a washing solution, an inlet conduit (2) of said washing solution in a fermenter (3), said fermenter (3) serving as propagation of the bacteria in a culture medium, an outlet conduit (4) for conveying the culture medium containing the bacteria to one or more tangential microfiltration modules (5), said modules (5) enabling separation of said culture medium into a permeate (6) not containing bacteria and a concentrate (7) containing the bacteria.

FIG. 1 illustrates the device according to the present invention.

According to the present invention, the concentrate (7) is recycled on leaving the filtration modules (5) by reincorporation into the fermenter (3).

According to the present invention, the filtration modules (5) comprise from 1 to 10 filtration membranes, each membrane representing from 0.1 m² to 150 m² total filtration surface and porosity between 0.01 and 0.5 μm, and preferably between 0.1 and 0.4 μm.

An object of the present invention is also a liquid concentrate of adapted and viable bacteria likely to be obtained by the process according to the present invention.

An object of the present invention is also utilisation of the liquid concentrate of adapted and viable bacteria, according to the present invention as food additive.

Foodstuff additive is understood to designate according to the present invention any chemical substance added to the foodstuffs during their preparation or in view of their storage to create a desired technical effect. In addition, according to the present invention, the liquid concentrate of bacteria has a stable numeration, the bacteria being viable and not causing fermentation in the finished additive product.

An object of the present invention is also an additive food product, characterised in that the foodstuff additive utilised is the liquid concentrate of adapted and viable bacteria according to the present invention.

According to the present invention, the food product is a milk product and/or a beverage.

Milk product is understood to designate according to the present invention, in addition to milk, products derived from milk, such as cream, iced cream, butter, cheese, yoghurt; secondary products, such as lactoserum, casein and various prepared foodstuffs containing milk or constituents of milk as principal ingredient.

Beverage is understood to designate according to the present invention beverages such as for example fruit juices, mixtures of milk and fruit juices, vegetable juices such as for example soy juice, oat juice or rice juice, alcoholic beverages such as for example kefir, sodas, and spring or mineral waters with added or not sugar or flavours, for example.

An object of the present invention is also a production process of a food additive product according to the present invention, characterised in that the liquid concentrate of adapted and viable bacteria is added to the food product at the end of the production line and preferably prior to packaging of the food product.

According to the present invention, the production process of a food additive product is characterised in that the liquid concentrate of adapted and viable bacteria is added to the food product in line by pumping.

The present invention will be better understood by means of the accompanying description to follow, which refers to examples of preparation of liquid concentrate of adapted and viable bacteria, according to the present invention.

It is understood, however, that these examples are given only by way of illustration of the object of the invention, whereof they could not otherwise constitute a limitation.

### FIGURES

FIG. 1 illustrates a device for concentration of bacteria by tangential filtration,

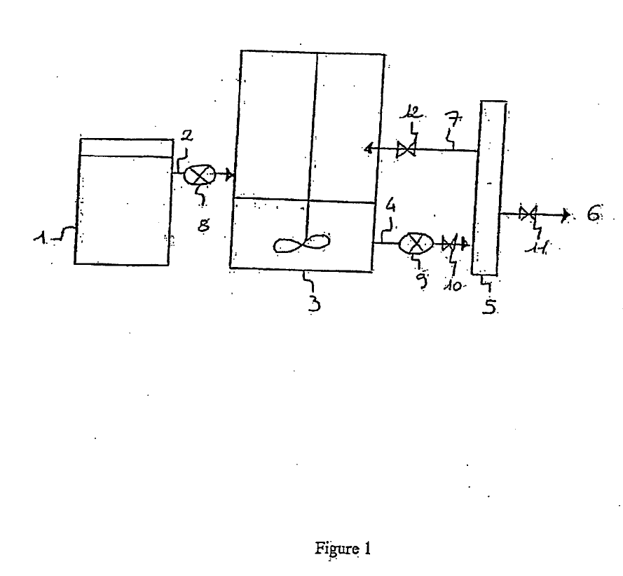

FIG. 2 illustrates the evolution of the transmembrane pressure over time,

FIG. 3 illustrates the evolution of the inlet pressure module,

FIG. 4 illustrates the evolution of the residue rates,

FIG. 5 illustrates the evolution of the optical density at 580 nm and of the transmembrane pressure.

### EXAMPLES

In these examples, the pressures are indicated in bars, one bar corresponding to 1.10⁵ Pa.

I. Revival and Preculture

Preparation of the Culture Medium

The starting culture medium is the MRS liquid medium (selective culture medium utilised for culture of the lactobacillus) without sugar in a bottle (95 ml), followed by sterile addition of our main source of carbon to produce 10 g/l, if it is a disaccharide or 20 g/l for a monosaccharide. Here 1 g of lactose is taken up in 5 ml warm distilled water, then the whole is filtered on a porosity filter of 0.2, um and added in totality to the 95-ml bottle of MRS. Ten ml are transferred to a sterile tube, intended for revival. The remainder (90 ml MRS at 10 g/l lactose) will be used for preculture.

Revival Growth Conditions (10 ml)
37° C.
in static in an oven
inoculation at 1% from a tube frozen at −80° C.
duration: 16 h
optic density measured on completion of culture on a sample diluted at 1/20 at 580 nm against a vat of water: 0.35 to 0.4
pH: close to 4

Preculture Growth Conditions (500 ml)
37° C.
in static in an oven
inoculation at 1% from the preculture
duration: 16 h
optic density measured on completion of culture on a sample diluted at 1/20 to 580 nm against a vat of water: 0.35 to 0.4
pH: close to 4.
   II. Propagation in Fermenter

Preparation of a Regulation Base of the pH

The KOH at 38% (or 9.3 mol/l) is utilised to neutralise the lactic acid product. It is sterilised at 121° C. for 15 minutes.

The prerequired volume for propagation of 10 litres is 1000 ml minimum.

Preparation of a Propagation Medium

The carbonated and nitrogenated sources are sterilised separately to avoid degradation reactions of the sugar (formation of Maillard compounds during sterilisation)

For 10 litres of final propagation medium:

Bottom of vat

| | |
|---|---|
| casein peptone tryptone (Merck) | 600 g |
| yeast extract (Merck) with HCl at 6 mol/l | 180 g |
| Adjust the pH to 6.5 | |
| MnSO₄, H₂O | 1 g |
| sqf 5.5 litres | |

Sterilise at 121° C. for 15 minutes in the fermenter previously sterilised with water.

Carbon source solution

Lactose 800 g

Dissolve hot then complete to 4 litres

Sterilise at 110° C. for 30 minutes Transfer this solution sterile hot to the bottom of the vat

Fermenter Propagation Conditions
Volume prior to inoculation: 9.5 litres.
37° C.
pH regulated to 6.5 with KOH 10 mol/l
duration: 18 h
agitation 200 rpm, agitation axle equipped with 3 immersed blades
degassing with nitrogen
permanent nitrogen feed from above (rate 11/minute)
inoculation at 5% from preculture or 500 ml
final optic density measured on completion of culture on un sample diluted at 1/100 to 580 nm against a vat of water: 0.32 to 0.35

After propagation, the result is a medium containing 2.10¹⁰ ufc/ml of bacteria.

III. Adaptation and washing of the culture

To prepare the biomass produced at pH, and/or at osmotic pressure and/or at the temperature of the finished product (yoghurt type) in which they will be injected, two steps are taken jointly:

After 17 hours of culture, a drop in pH is made by natural acidification in one hour to go from pH 6.5 to pH 5 (=pH target before washing).

Washing the bacteria (at 37° C.) is done after the batch (after the acidification step at pH 5).

Washing is done in a solution of saccharose at 250 g/l, sterilised for 30 minutes at 100° C., corresponding to a solution of osmotic pressure of 1000 mOsm. This choice is optimised to minimise the risks of osmotic shock in going from a synthetic medium to the finished product whereof the measured parameters are pH 5 and an osmotic pressure of 879 mOsm.

The steps during washing are startup of the filtration loop, recirculation of the bacteria through the system and injection of the washing solution/removal of the filtrate at the same rate.

Startup of the Filtration System

During filtration startup, the first step is formation of the polarisation layer by having the system running for 5 minutes at reduced speed (20 to 50% of the maximum rate of the pump) with the inlet and outlet valves of the module in an open position at 100%. The permeate outlet valve is closed. Once this period passes the rating of the pump is progressively increased to 100% of its range of use. The permeate valve is open to 100% and kept in this position for the entire filtration step.

To maintain a constant volume of reactive medium during washing, the permeation volume must be equivalent to that of the supply (D1). The supply rate of the washing solution is identical to that of the permeate. The volume of the solution is passed in a period varying between 1 and 2 hours. Once this period is past, the filtration conditions remain unchanged, and volume concentration begins.

IV. Concentration by Tangential Microfiltration.

Filtration of the medium is effected in a temperature range between 25 and 44° C. at the target pH between 3.5 and 5.5 over 4 hours. The filtration rate drops sharply with the advance of the culture and the modifications to the rheological characteristics of the medium. The inlet pressure of the loop increases to reach a value of 3 bars, representing the upper limit supported by the filtration membranes. Recirculation of the medium is then stopped and the bacterian concentrate is recovered sterile. This method produces a litre of creamy liquid containing at least 1.10¹¹ ufc/ml of bacteria.

Operating Conditions

1. Sterilisation of the Complete System.

the filtration loop is sterilised by passage of flowing steam. To carry out this operation, the filtration module must be equipped with dilation compensation screws.

The efficacy of the treatment is evaluated by calculating the sterilising value Fo. This is the time in minutes of a sterilisation reckoner having the same efficacy at the reference temperature 121.1° C.

Either t, the time of treatment, or T the temperature of treatment, with z=10° C. as per international convention.
Fo=t.10^{(T-Tref/z)}

Two passes of sterile water (121.1° C. for 20 min) originating from the fermenter, are made in the loop prior to filtration of the bacteria.

2. Cleaning and Recycling of Membranes.

Recovery of the membranes after filtration is easy but must follow the reference variables described hereinbelow:

treatment by a solution of NaOH 0.5 M at 45° C. for 30 minutes, pump at maximum rate before rinsing. Thus treated, the membranes can be reutilised for at least 5 reproducible production cycles of concentrated L. casei.

3. Storing Membranes on Site.

The whole module is conserved with a solution of NaOH 0.1 M, with all valves closed, if necessary.

4. Startup of the Microfiltration Step.

One of the major risks when using the microfiltration technique is substantial and rapid clogging of the membrane.

This clogging is characterised by three phenomena:
adsorption and adhesion of particles and solutes on the membrane surfaces
polarisation layer and formation of a cake
blocking of pores

As a general rule, weak transmembrane pressure as well as high tangential circulation speed are fundamental parameters in the execution of this operation.

III Characterisation of the Platform.

1. Measuring Parameter.

The range of measuring is made over a duration of 300 minutes on average.

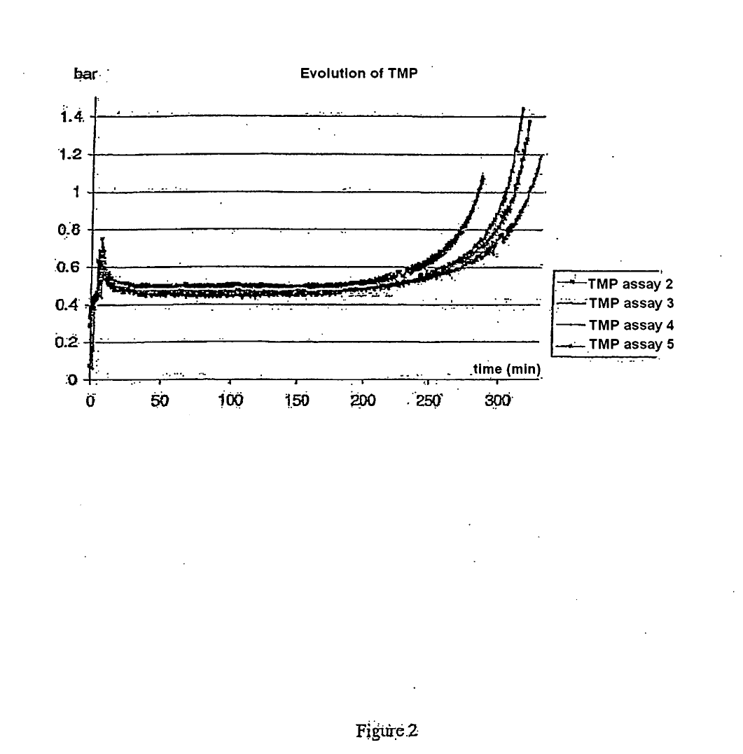

With reference to the assays carried out (see FIG. 2) the time range between 15 and 175 minutes presents a stability phase of inlet and outlet pressure module, and consequently of the transmembrane pressure.

This range of 160 minutes is representative of optimum filtration conditions to be maintained.

The tables below represent the values measured at the outset, the middle and at the end of filtration time.
1.1 Evolution of Pressures

The propellant of selective separation on a porous membrane is the differential in pressure existing between the residue circuit and the permeate circuit.

TABLE 1
Evolution of pressures

| | | | | | | | |
|---|---|---|---|---|---|---|---|
| | P inlet | Δ/ | P outlet | Δ/ | | Δ/ | |
| ASSAY | Bar | P initial | bar | P initial | P permeate | P initial | TMP |

Assay 1

| | | | | | | | |
|---|---|---|---|---|---|---|---|
| Initial pressure | 1.245 | / | 0.119 | / | 0.166 | / | 0.528 |
| Pt = 150 min | 1.223 | −0.022 | 0.144 | 0.025 | 0.145 | −0.032 | 0.482 |
| Pt = 295 min | 2.382 | 1.137 | 0.2I2 | 0.093 | 0.206 | 0.04 | 1.062 |
| P final | 2.415 | 1.170 | 0.218 | 0.099 | 0.216 | 0.05 | 1.075 |
| t = 295.5 min | | | | | | | |

Assay 2

| | | | | | | | |
|---|---|---|---|---|---|---|---|
| Initial pressure | 1.187 | / | 0.117 | / | 0.164 | / | 0.493 |
| Pt = 150 min | 1.199 | 0.012 | 0.143 | 0.026 | 0.218 | 0.054 | 0.454 |
| Pt = 300 min | 2.309 | 1.122 | 0.175 | 0.058 | 0.252 | 0.088 | 0.928 |
| P final | 3.179 | 1.992 | 0.275 | 0.158 | 0.299 | 0.135 | 1.426 |
| t = 315 min | | | | | | | |

Assay 3

| | | | | | | | |
|---|---|---|---|---|---|---|---|
| Initial pressure | 1.230 | / | 0.118 | / | 0.134 | / | 0.518 |
| Pt = 150 min | 1.207 | −0.023 | 0.145 | 0.027 | 0.220 | 0.086 | 0.454 |
| Pt = 300 min | 1.852 | 0.622 | 0.137 | 0.019 | 0.231 | 0.097 | 0.769 |
| P final | 2.734 | 1.504 | 0.253 | 0.135 | 0.268 | 0.134 | 1.188 |
| t = 328 min | | | | | | | |

Assay 4

| | | | | | | | |
|---|---|---|---|---|---|---|---|
| Initial pressure | 1.217 | / | 0.121 | / | 0.160 | / | 0.512 |
| Pt = 150 min | 1.197 | −0.02 | 0.I48 | 0.027 | 0.199 | 0.039 | 0.472 |
| Pt = 300 min | 2.045 | 0.828 | 0.153 | 0.032 | 0.217 | 0.057 | 0.834 |
| P final | 3.009 | 1.792 | 0.285 | 0.164 | 0.258 | 0.098 | 1.371 |
| t = 318.5 min | | | | | | | |

measuring of transmembrane pressure (TMP)
TMP=(P_{module entry}+P_{module outlet})/2)−Pₚₑᵣₘₑₐₜₑ

With inlet pressure module equal to recirculation pressure

After a 15-minute cycle we consider the whole of the system to be stabilised. The polarisation layer is then established, and overall pressures and rates are stabilised.

Average measured value during assays over the range stabilised measurement:

| | |
|---|---|
| Inlet pressure of the module: | 1.211 bar |
| Outlet pressure of the module: | 0.145 bar |
| Permeate pressure: | 0.196 bar |
| Evolution of the TMP | 0.465 bar |

1.2. Evolution of Rates.

TABLE 2
Evolution of rates and tangential speed

| | | | | | Circulation |
|---|---|---|---|---|---|
| | Inlet | | Q permate | | speed |
| ASSAY | 1/h | m³/h | 1/h | m³/h | m/s |

Assay 1

| | | | | | |
|---|---|---|---|---|---|
| Initial rate | 108.5 | 0.1085 | 2.22 | 0.00222 | 0.502 |
| Q t = 150 min | 127.1 | 0.1271 | 1.43 | 0.00143 | 0.588 |
| Q t = 295 min | 13.1 | 0.0131 | 0.99 | 0.00099 | 0.061 |
| Q final t = 295.5 min | 11.4 | 0.0114 | 0.97 | 0.00097 | 0.053 |

Assay 2

| | | | | | |
|---|---|---|---|---|---|
| Initial rate | 107.3 | 0.1073 | 2.03 | 0.00203 | 0.497 |
| Q t = 150 min | 124.5 | 0.1245 | 1.44 | 0.00144 | 0.576 |
| Q t = 300 min | 66.3 | 0.0663 | 1.32 | 0.00132 | 0.307 |
| Q final t = 315 min | 20.7 | 0.02067 | 1.03 | 0.00103 | 0.096 |

Assay 3

| | | | | | |
|---|---|---|---|---|---|
| Initial rate | 101.6 | 0.10159 | 2.32 | 0.002323 | 0.470 |
| Q t = 150 min | 122.8 | 0.12276 | 1.52 | 0.001516 | 0.568 |
| Q t = 300 min | 93.5 | 0.0935 | 1.41 | 0.001406 | 0.433 |
| Q final t = 328 min | 43.3 | 0.04326 | 1.48 | 0.001483 | 0.200 |

Assay 4

| | | | | | |
|---|---|---|---|---|---|
| Initial rate | 107.2 | 0.1072 | 2.18 | 0.00218 | 0.496 |
| Q t = 150 min | 124.7 | 0.1247 | 1.55 | 0.00155 | 0.577 |
| Q t = 300 min | 83.2 | 0.0832 | 1.41 | 0.00141 | 0.385 |
| Q final t = 318.5 min | 29.3 | 0.0293 | 1.37 | 0.00137 | 0.136 |

linear speed (m/s)

Vt=Q(m³/h)/ (3600×total filtration surface) in m/s

With total filtration surface number of modules x number of channels x section (in m)

Over the range of stabilised measurement:

| | | |
|---|---|---|
| Average measured maximum residue rate: | 124.8 | l/h |
| Maximum permeate rate | 2.19 | l/h |
| Average permeate rate: | 1.46 | l/h |
| Average tangential speed: | 0.579 | m/s |

1.3. Evolution of Temperature.

Throughout all our assays the maximum measured elevation relative to instructions was 2° C.

A thermal changer placed at the pump outlet or module could easily contain this rise in temperature. In general, the temperature measured is constant at 37° C. with measuring spread of 0.3° C.
1.4. Concentration Factors

The volume concentration factor (VCF) is 10.

The final population achieved in batch is 2.10¹⁰ ufc/ml. The final population measured in the bacterian concentrate is greater than 1.5.10¹¹ ufc/ml.
1.5. Reproducibility of the Filtration Operation

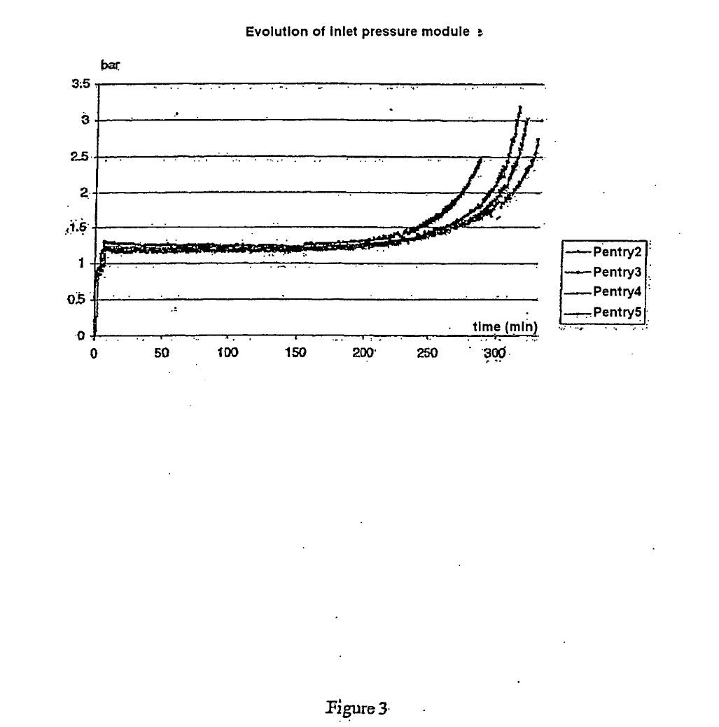

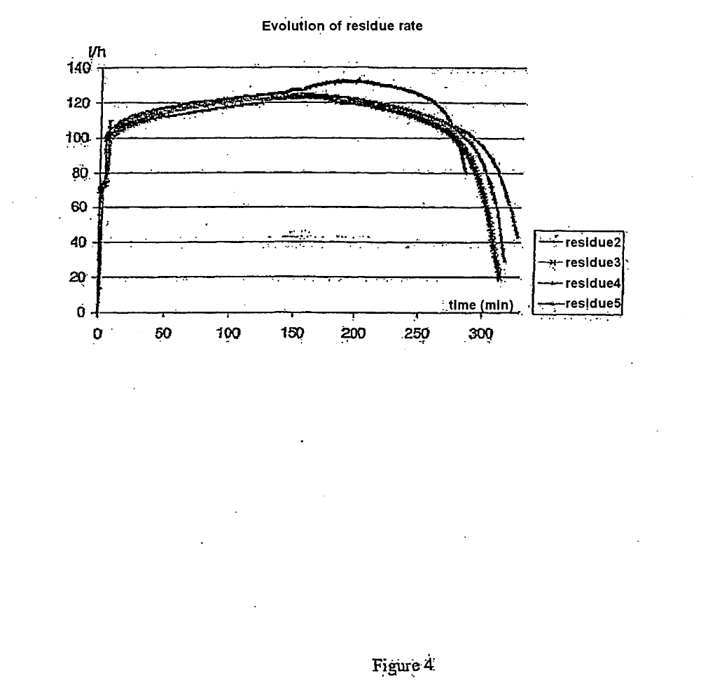

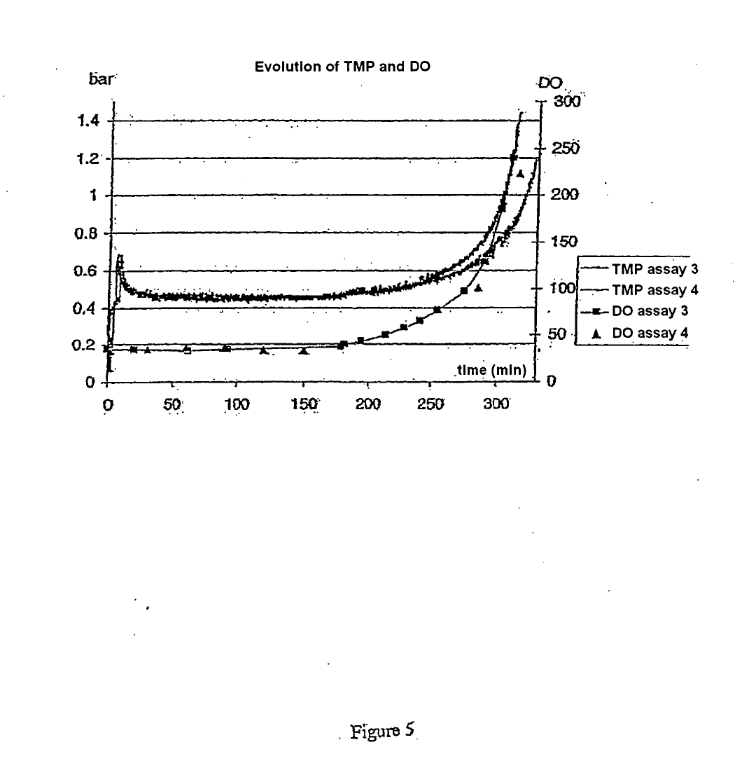

This is evaluated by tracing curves appearing in FIGS. 3 to 5 hereinafter, for the different filtration assays conducted over 4 weeks during the mouse test.

The tangential filtration step in the conditions described is perfectly reproducible, and the parameters of rate, temperature and pressure are controlled during the concentration step of L. casei.

Tangential filtration platform: Conditions for obtaining a final population of L. casei of 1.10¹⁰ ufc/ml.
General Conditions:

Population end of batch: 2.10¹ ufc/ml

Washing bacteria in a saccharose solution (250 g/l) osmotic pressure 1000 mOsm.

(injection by pump/removal by filtration at the same rates: 66 ml/min): duration 1 h30

Duration of filtration: duration 4 h

TABLE 3
Average rates

| | | |
|---|---|---|
| Residue | 125 | l/h |
| Permeate | 1.50 | l/h |

Average pressures

| | | |
|---|---|---|
| Inlet | 1.21 | bar |
| Outlet | 0.14 | bar |
| Permeate | 0.19 | bar |
| TMP | 0.46 | bar |

Temperature
37° C.
Average tangential speed
0.580 m/s

1. A production process for a liquid concentrate of adapted and viable bacteria, for use in foodstuffs comprising the following successive steps:
a) the bacteria are propagated in a fermenter in an appropriate culture medium;
b) the bacteria obtained are adapted to step a);
c) the culture medium containing the bacteria adapted by tangential microfiltration is washed using a washing solution;
d) the washed medium containing the bacteria adapted by tangential microfiltration to a bacterial concentration greater than 5.10¹⁰ ufc/ml advantageously greater than 1.10¹¹ ufc/ml are concentrated in bacteria;
e) a liquid concentrate of adapted and viable bacteria for use in foodstuffs is recovered.
and/or adaptation of the bacteria carried out at step b) is disclosed by measuring parameters of the culture medium and/or bacteria parameters.

2. The process as claimed in claim 1, wherein the bacteria are lactic bacteria, in particular bacteria of Lactobacillus spp, Bifidobacterium spp., Streptococcus spp and Lactococcus spp genera.

3. The process as claimed in claim 1, wherein the culture medium of step a) is a synthetic medium.

4. The process as claimed in claim 1, wherein the culture medium containing the bacteria in the fermenter at the end of step a) has a pH between 3 and 6.

5. The process as claimed in claim 1, wherein the concentration of bacteria at the end of propagation step a) is greater than 2.10¹⁰ ufc/ml.

6. The process as claimed in claim 1, wherein the parameters of the culture medium are the pH, the osmotic pressure and/or the temperature of the culture medium.

7. The process as claimed in claim 6, wherein the parameter of the culture medium is the pH and in that the step b) is taken by reducing the pH by natural acidification.

8. The process as claimed in claim 6, wherein the parameter of the culture medium is the temperature, and in that step b) is taken by reducing the temperature.

9. The process as claimed in claim 1, wherein the parameter of the bacteria is the size of the bacteria.

10. The process as claimed in claim 1, wherein the distribution of the lengths of each bacterium is predominantly between 0.1 and 10 micrometres, advantageously between 0.5 and 5 micrometres.

11. The process as claimed in claim 1, wherein adaptation step b) is taken by tangential microfiltration.

12. The process as claimed claim 1, wherein the tangential microfiltration membranes have a porosity between 0.01 and 0.5 μm, advantageously between 0.1 and 0.3 μm.

13. The process as claimed in claim 1, wherein in step c) the inlet pressure of the culture medium in the microfiltration module is between 0 and 3. 10⁵ Pa.

14. The process as claimed in claim 1, wherein in steps c) and d) the rate of the permeate is between 0.001 and 0.1 m³/h/m² of surface exchange.

15. The process as claimed in claim 1, wherein in step d) the transmembrane pressure is between 0.1.10⁵ and 2.10⁵ Pa and advantageously between 0.1.10⁵ and 0.5.10⁵ Pa.

16. The process as claimed in claim 1, wherein in step d) the recirculation rate of the washed medium is between 0.5 and 3 m³/h/m² of exchange surface and advantageously between 0.8 and 1.25 m³ /h/m² of exchange surface.

17. The process as claimed in claim 1, further comprising prior to step a) successive steps of revival and preculture of the bacteria.

18. The process as claimed in claim 1, further comprising an additional step f), following step e), of packaging the liquid concentrate of adapted and viable bacteria in flexible and hermetic bags.

19. The process as claimed in claim 18, further comprising an additional step g), following step f), of keeping the liquid concentrate of adapted and viable bacteria packaged in flexible bags and hermetic at a temperature between −50° C. and +4° C.

20. The process as claimed in claim 19, further comprising an additional step h), following step g), of reheating by adapted means of the liquid concentrate of adapted and viable bacteria packaged in flexible and hermetic bags.

21. A device for executing the process for production of a liquid concentrate of adapted and viable bacteria for use in foodstuffs as claimed in claim 1, comprising a vat (1) containing a washing solution, an inlet conduit (2) of said washing solution in an fermenter (3), said fermenter (3) serving as propagation of the bacteria in a culture medium, an outlet conduit (4) for conveying the culture medium containing the bacteria to one or more modules (5) of tangential microfiltration, said modules (5) allowing separation of said culture medium into a permeate (6) not containing bacteria and into a concentrate (7) containing the bacteria.

22. The device as claimed in claim 21, wherein the concentrate (7) is recycled on leaving the filtration modules (5) by reincorporation into the fermenter (3).

23. The device as claimed in claim 21, wherein the filtration modules (5) comprise from 1 to 10 filtration membranes, each membrane representing from 0.1 m² to 150 m² of total filtration surface.

24. A liquid concentrate of adapted and viable bacteria, characterized in that it is likely to be obtained by the process as claimed in claim 1.

25. A foodstuff comprising the liquid concentrate of adapted and viable bacteria as claimed in claim 24.

26. A food product additive comprising a liquid concentrate of adapted and viable bacteria as claimed in claim 24.

27. A milk product and/or beverage comprising the food product additive as claimed in claim 26.

28. A manufacturing process for an additive food product as claimed in claim 26, wherein the liquid concentrate of adapted and viable bacteria is added to the food product at the end of the production line and preferably prior to packaging of the food product.
